(19) United States
(12) Patent Application Publication
(10) Pub. No.: US 2018/0296150 A1
(43) Pub. Date: Oct. 18, 2018

(54) METHOD AND DEVICE FOR TESTING USER EXPERIENCE

(71) Applicant: AAC Technologies Pte. Ltd., Singapore city (SG)

(72) Inventors: Yao Wang, Shenzhen (CN); Rui Wu, Shenzhen (CN); Rongguan Zhou, Shenzhen (CN); Grigori Evreinov, Tampere (FI); Ahmed Farooq, Tampere (FI); Philipp Weitz, Tampere (FI); Roope Raisamo, Tampere (FI); Arto Hippula, Tampere (FI); Timo Lamberg, Tampere (FI)

(21) Appl. No.: 15/862,998

(22) Filed: Jan. 5, 2018

(30) Foreign Application Priority Data
Apr. 13, 2017 (CN) 201710239055.1

(51) Int. Cl. A61B 5/16; A61B 5/00

(52) U.S. Cl. CPC A61B 5/162; A61B 2560/0223; A61B 5/486; A61B 5/0051

(57) ABSTRACT

A method for testing user experience, comprising steps of: acquiring test indicating information; releasing a main task and an auxiliary task according to the test indicating information, each auxiliary task includes the main task and a corresponding physical signal providing prompting effect or inhibiting effect to the main task; acquiring feedback response information of a subject corresponding to the main task and the auxiliary task. In the technical solution of the present disclosure, through comparison of feedback response information of the subject between the auxiliary task and the main task, the perception situation of the subject on physical signals can be determined, and through comparison of feedback response information of the subject between the auxiliary tasks, the perception situation of the subject on different physical signals can be determined, thereby improving the effectiveness of the test result on perception attributes of physical signals.

### TECHNICAL FIELD

The present disclosure related to the field of somatosensory testing and evaluating and, in particular, to a method and a device for testing user perception.

### BACKGROUND ART

The brain of a human will automatically and effectively converge information while receiving the information from the environment so as to acquire information, and perform internal representation, which generates feeling and perceptual experience for a human. When the brain is stimulated during receiving information from the environment, the perceptual information acquired by the brain will be changed, which means, new information is received and the internal representation is updated. The stimulation here refers to a promoting process or an inhibiting process. Such a perception test technique has been used in various researches on human factor psychology.

For example, the perceptual intensity of visual stimulation can be strengthened or weakened through acoustic stimulation which interacts at the threshold stimulus level, that is, the effect of perception strengthened or inhibited will happen depending on sensory modality. Specifically, when the brain is receiving target information delivered with a modality specific stimulus, a neural response A will be generated. However, if the brain is receiving target information, while affected by distractor such as a sound of talking by a neighbor or sound of taking off of an airplane is received, a neural response B will be generated. Since the sound of talking by the neighbor or the sound of taking off of the airplane and the original target information will bring cross action to the brain of a human, the original neural response A will be inhibited or prompted, so that the neural response B will be obtained. Through using the difference between the neural response A and neural response B, the perceptual attributes of a human on physical signals can be obtained.

At present, when testing the perceptual attributes of a human on physical signals, the physical parameter weight of vibration is acquired based on subjective sensation of subjects. The experience, attitude, impression etc. of the subjects on vibration signals will influence the data processing of the subjects, which may cause inaccuracy of the test results of the perceptual attributes on physical signals.

Therefore, it is an urgent technical problem to be solved that, how could the above perception test technique be applied to perception test on physical signals, so as to improve effectiveness of the test results of the perceptual attributes on physical signals.

### BRIEF DESCRIPTION OF DRAWINGS

Many aspects of the exemplary embodiment can be better understood with reference to the following drawings. The components in the drawings are not necessarily drawn to scale, the emphasis instead being placed upon clearly illustrating the principles of the present disclosure. Moreover, in the drawings, like reference numerals designate corresponding parts throughout the several views.

FIG. 1 is a flow diagram of a method for testing user experience in accordance with an exemplary embodiment of the present disclosure;

FIG. 2A is a schematic diagram of a testing principle of a main task;

FIG. 2B is a schematic diagram of a testing principle of an auxiliary task;

FIG. 3 is a flow diagram for completing the test;

FIG. 4 is a flow diagram of a method for testing user experience in accordance with another exemplary embodiment of the present disclosure;

FIG. 5 is a block diagram of a device for testing user experience in accordance with an exemplary embodiment of the present disclosure; and

FIG. 6 is a block diagram of a testing system in accordance with an exemplary embodiment of the present disclosure.

### DESCRIPTION OF EMBODIMENT

In order to better understand technical solutions of the present disclosure, the present disclosure will be described in detail with reference to the drawings.

It should be noted that, the embodiments described are only partial embodiments of the present disclosure, rather than all of the embodiments. Based on the embodiments in the present disclosure, other embodiments obtained by those skilled in the art without creative work are all belonging to the protection scope of the present disclosure.

The terms used in the embodiments of the present disclosure is only used for the objective of describing specific embodiments, which is not used to limit the present disclosure. The singular form of ‘a’, ‘an’, ‘the’ and ‘said’ used in the embodiments of the present disclosure and the claims is intended to include the multiple form, unless otherwise clearly indicated according to the context.

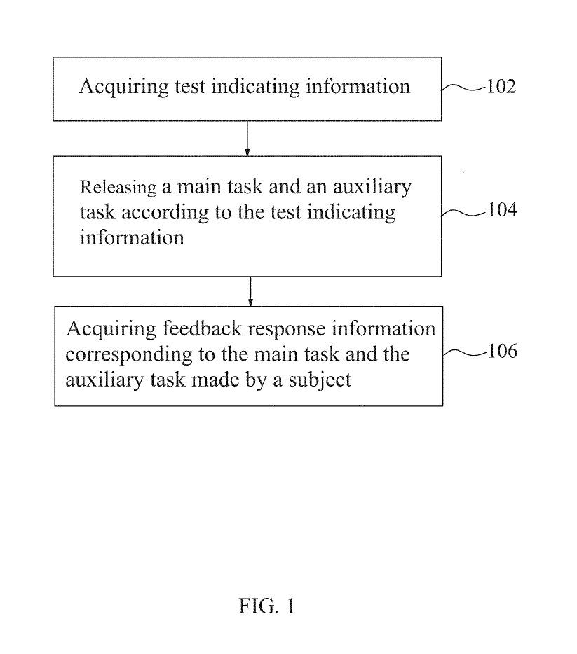

FIG. 1 is a flow diagram of a method for testing user experience in accordance with an exemplary embodiment of the present disclosure.

As shown in FIG. 1, a method for testing user experience in accordance with an exemplary embodiment of the present disclosure, including:

Step 102, acquiring test indicating information.

Step 104, releasing a main task and an auxiliary task according to the test indicating information, each auxiliary task includes the main task and a corresponding physical signal providing prompting effect or inhibiting effect to the main task.

The main task refers to a basic main task, and the auxiliary task refers to a main task incorporated with a corresponding physical signal. Different physical signals correspond to different auxiliary tasks, thus there can be one or more auxiliary task.

Step 106, acquiring feedback response information corresponding to the main task and the auxiliary task made by a subject.

After the system releases a task, the subject can input corresponding feedback response information for the task through a touching action. Since the auxiliary signal includes an additional physical signal compared with the main task, the physical signal can generate prompting effect or inhibiting effect to the main task. Therefore, through the comparison of the feedback response information corresponding to the auxiliary task and the main task made by the subject, perception situations to physical signals by the subject can be determined. Through the comparison of the feedback response information corresponding to different auxiliary tasks made by the subject, perception situations to different physical signals by the subject can be determined.

In conclusion, the effectiveness of the perception test results on physical signal attributes is improved, and effective test results are acquired.

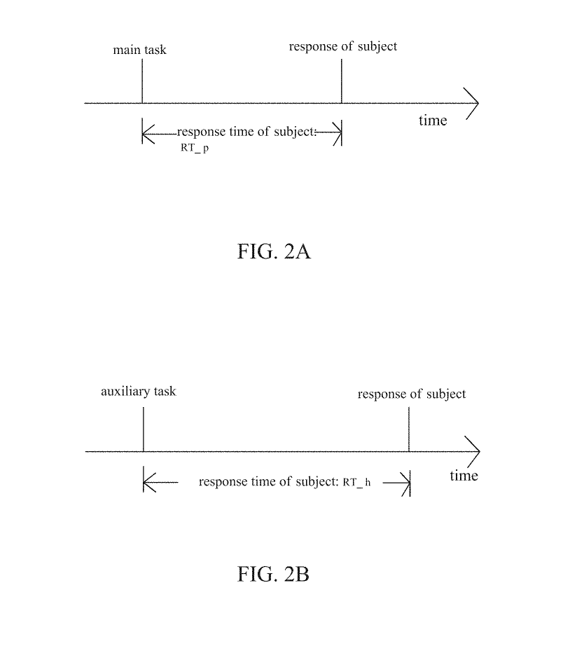

FIG. 2A is a schematic diagram of a testing principle of a main task.

As shown in FIG. 2A, the main task is released at a time point. The main task can be a visual stimulation or acoustic stimulation or other forms of stimulation,

The subjects are required to focus on a visual stimulation, such visual simulation requires the subjects to be quite close to the background screen of the computer, and the stimulation will appear only for a quite short period of time. As a result, it is guaranteed that a high perceptual overload is required so as to complete the main task. That is to say, once the subject sees the visual stimulation, the subject shall press the touch button immediately. The touch button can be a non-deformable button on the glass plate or screen of a cellphone having a sensor. In this way, the system records the time point of pressing the touch button, i.e., the time point at which a response is made by the subject.

The time difference between the time point of releasing the main task and the time point of making a response by the subject is the response time of the subject.

Of course, the main task is not limited to visual stimulation, which can also be acoustic stimulation and the like.

FIG. 2B is a schematic diagram of a testing principle of an auxiliary task.

As shown in FIG. 2B, the auxiliary task is released at a time point. The auxiliary task includes the main task and a physical signal corresponding to the main task. The main task can be a visual stimulation, and the physical signal includes but is not limited to touch stimulation, which can also be acoustic signals, electric signals, optical signals etc. A touch stimulation may be implemented by a random vibration through a motor fixed on the glass plate, that is to say, the physical signal herein is vibration.

Once the subject sees the visual stimulation, the subject shall press the touch button immediately. The touch button can be a non-deformable button on the glass plate or an area of screen of a cellphone having a sensor. In this way, the system records the time point of pressing the touch button, i.e., the time point at which a response is made by the subject. However, comparing with FIG. 2A, it can be seen that, since a physical signal is incorporated, inhibiting effect or prompting effect is generated to perceptual ability of the subject, thus the response time point of the subject is later than or previous to that in FIG. 2A. As a result, the response time of the subject in FIG. 2B is longer or shorter than the subject in FIG. 2A. Of course, during this process, not only the response time of the subject is recorded by the system, but also various feedback response information made by the subject can be recorded, such as pressing pressure or pressing time and the like.

Through the manner of detecting the strengthening or inhibition and acquiring feedback response information of the subject, the motor vibration parameter such as displacement, acceleration, frequency and the like can be evaluated, so as to obtain a more reliable test result.

Of course, the main task is not limited to visual stimulation, which can also be acoustic stimulation and the like.

Based on the above principle, when a subject has only completed the main task, the response time is recorded as RT_p. When the main task and the auxiliary task are released to the subject at the same time, the response time of the subject is recorded as RT_h. Theoretically, the physical signals may prompt or inhibit the subject in completing the main task, that is, RT_p may be larger than RT_h, or smaller than RT_h.

In an exemplary embodiment of the present disclosure, step 104 specifically includes: releasing, based on a predetermined releasing rule or randomly, a main task and an auxiliary task according to the test indicating information. The number of the main task is larger than the number of the auxiliary task, so that the subject can be focused on the main task, so as to acquire more accurate measurement data.

In an exemplary embodiment of the present disclosure, the auxiliary task includes a first auxiliary task and a second auxiliary task. The physical signal of the first auxiliary task and the physical signal of the second auxiliary task have at least one difference parameter; and the method includes, after step 106, determining perception situation of the subject on the difference parameter according to the feedback response information of the subject.

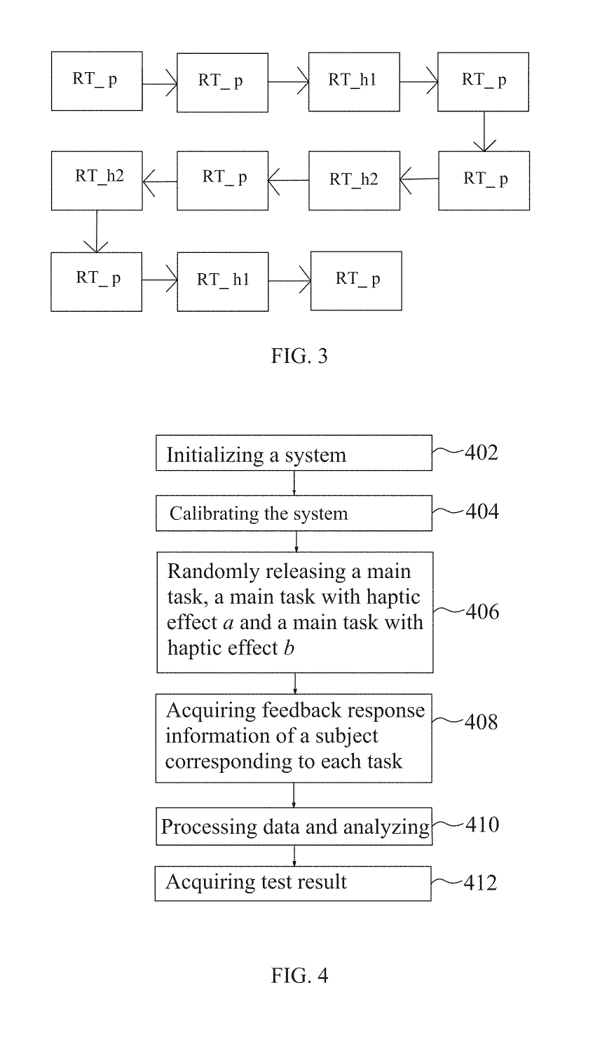

In specific scenario, in the flow diagram shown in FIG. 3, RT_p represents a response time of a subject after sensing the main task until a response is made, RT_h1 represents a response time of a subject after sensing the main task and a haptic vibration effect a until a response is made. RT_h2 represents a response time of a subject after sensing the main task and a haptic vibration effect b until a response is made.

During the test, the main tasks are the majority, the main task with haptic vibration effect a and the main task with haptic vibration effect b appear randomly, and the number of which is not much.

Taking RT_p as a standard, through data processing, the difference of perception on two vibration effect haptic a and haptic b can be acquired, and which haptic the subject is more sensitive to can be acquired. Furthermore, the perception situations of the subject on specific parameter can be studied. When most of the parameters of the two haptics are the same, the perception situation of the variable parameter can be studied.

The above contents will be described in further detail with reference to a method for testing user experience shown in FIG. 4.

As shown in FIG. 4, the implementing process of the method for testing user experience includes:

Step 402, initializing a system.

Step 404, calibrating the system. The calibration item includes but is not limited to: system configuration parameter, such as calibrating the touching perception level of the calibrating system, calibrating the perceptual overload of the subject and the like.

Step 406, randomly releasing a main task, a main task with haptic effect a and a main task with haptic effect b.

The number of the main task is larger than the number of the main task with haptic effect a, and is also larger than the number of the main task with haptic effect b. The total number of the tasks is from 300 to 400. Of course, the number is not limited to this range.

Step 408, acquiring feedback response information of a subject corresponding to each task.

The acquiring step is performed during the task or after the task. Obviously, it is possible to record the tasks all together and record the feedback response information of the subject all together, and targeting the recorded task and the feedback response information correspondingly according to time information.

The feedback response information of the subject includes but is not limited to at least one of response time, pressing pressure and pressing time.

Step 410, processing data and analyzing. The perception difference of the subject on two different vibration effects is determined.

Step 412, acquiring test result. That is, which vibration effect the subject is more sensitive to is acquired. Furthermore, the perception situations of the subject on specific parameter can be studied. When most of the parameters of the two haptics are the same, the perception situation of the variable parameter can be studied.

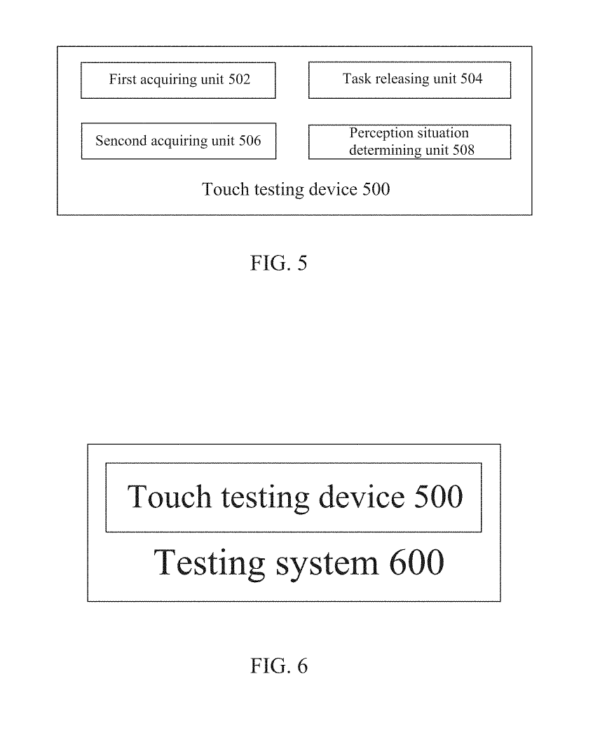

FIG. 5 is a block diagram of a device for testing user experience in accordance with an exemplary embodiment of the present disclosure.

As shown in FIG. 5, an exemplary embodiment of the present disclosure provides a device 500 for testing user experience, the touch testing device 500 shown in FIG. 5 adopts the method for testing user experience according to any one of the above embodiments, and thus possesses all the technical effects above, which will not be repeated herein.

The device 500 for testing user experience includes: a first acquiring unit 502, a task releasing unit 504 and a second acquiring unit 506.

The first acquiring unit 502 is configured to acquire test indicating information.

The task releasing unit 504 is configured to release a main task and an auxiliary task according to the test indicating information, each auxiliary task includes the main task and a corresponding physical signal providing prompting effect or inhibiting effect to the main task.

The second acquiring unit 506 is configured to acquire feedback response information of subject corresponding to each task.

In an exemplary embodiment of the present disclosure, optionally, the task releasing unit 504 is specifically configured to release, based on a predetermined releasing rule or randomly, a main task and an auxiliary task according to the test indicating information. The number of the main task is larger than the number of the auxiliary task.

In an exemplary embodiment, optionally, the physical signal is vibration.

In an exemplary embodiment, optionally, the feedback response information of the subject includes pressing time, or further includes one or more of response time and pressing pressure.

In the present embodiment of the present disclosure, optionally, the auxiliary task includes a first auxiliary task and a second auxiliary task. The physical signal of the first auxiliary task and the physical signal of the second auxiliary task have at least one difference parameter. The touch testing device 500 further includes: a perception situation determining unit 508, configured to, after the second acquiring unit 506 acquires the feedback response information of the subject, determine perception situation of the subject on the difference parameter according to the feedback response information of the subject.

FIG. 6 is a block diagram of a testing system in accordance with an exemplary embodiment of the present disclosure.

As shown in FIG. 6, a testing system 600 is provided according to an exemplary embodiment of the present disclosure, which includes the touch testing device 500 shown in FIG. 5. Therefore, the testing system 600 has the same technical effect with the touch testing device 500 shown in FIG. 5.

The technical solution of the present disclosure is illustrated as above in great detail with reference to the drawings. Therefore, through the comparison of the feedback response information corresponding to the auxiliary task and the main task made by the subject, perception situations to physical signals by the subject can be determined. Through the comparison of the feedback response information corresponding to different auxiliary tasks made by the subject, perception situations to different physical signals by the subject can be determined. In conclusion, the effectiveness of the perceptual test results on physical signal attributes is improved, and effective test results are acquired.

Depending on the context, the expression ‘if’ used herein can be interpreted as ‘when’ or ‘in response to determination’ or ‘in response to detection’. Similarly, depending on the context, phrase ‘if . . . is determined’ or ‘if . . . is detected’ can be interpreted as ‘when it is determined that’ or ‘in response to determination’ or ‘when it is detected that’ or ‘in response to detection’.

In the embodiments provided by the present disclosure, it should be understood that, the disclosed system, device and method can be implemented through other manners. For example, the above-mentioned device embodiments are only exemplary, for example, the division of units is merely a logical functional division. In practical application, there may be other division manners, for example, a plurality of units or components can be combined or integrated into another system, or some characteristics can be ignored, or not executed. In addition, the mutual coupling or direct coupling or communication connection shown or discussed can be implemented through indirect coupling or communication connection through some interfaces, devices or units, which can be electrical, mechanical or in other forms.

Besides, each function unit in each embodiment of the present disclosure can be integrated in a processing unit, or each unit can physically be provided in individual, or two or more units can also be integrated in one unit. The above integrated unit can be implemented by hardware, or can be implemented by hardware plus software function unit.

The above-mentioned unit implemented in form of software function unit can be stored in a computer readable storage medium. The above software function unit is stored in a storage medium, including a number of instructions used to make a computer device (e.g. a personal computer, a server, or a network device and so on), or a processor to execute partial steps of the method of each embodiment of the present disclosure. However, the above-mentioned storage mediums include: USB flash disk, mobile hard disk drive, read-only memory (ROM), random access memory (RAM), disk or optical disk and the like which can store programming code.

The above are merely the preferred embodiments of the present disclosure, which will not limit the present disclosure. Any modifications, equivalent replacements and improvements made within the spirits and principles of the present disclosure shall all fall in the protection scope of the present disclosure.

What is claimed is:

1. A method for testing user experience, comprising steps of:
acquiring test indicating information;
releasing a main task and an auxiliary task according to the test indicating information, the auxiliary task includes the main task and a corresponding physical signal providing prompting effect or inhibiting effect to the main task;
acquiring feedback response information of a subject corresponding to the main task and the auxiliary task.

2. The method for testing user experience as described in claim 1, wherein the step of releasing a main task and an auxiliary task according to the test indicating information, specifically comprises:
releasing, based on a predetermined releasing rule or randomly, a main task and an auxiliary task according to the test indicating information, wherein a number of the main task is larger than a number of the auxiliary task.

3. The method for testing user experience as described in claim 1, wherein
the physical signal is a vibration signal.

4. The method for testing user experience as described in claim 1, wherein
the feedback response information of the subject comprises response time.

5. The method for testing user experience as described in claim 4, wherein the feedback response information of the subject further comprises at least one of pressing pressure and pressing time.

6. The method for testing user experience as described in claim 4, wherein the auxiliary task comprises a first auxiliary task and a second auxiliary task, a physical signal of the first auxiliary task and a physical signal of the second auxiliary task have at least one difference parameter; and
after acquiring feedback response information of a subject corresponding to the main task and the auxiliary task, further comprising:
determining perception situation of the subject on the difference parameter according to the feedback response information of the subject.

7. A device for testing user experience, comprising:
a first acquiring unit, configured to acquire test indicating information;
a task releasing unit, configured to release a main task and an auxiliary task according to the test indicating information, each auxiliary task includes the main task and a corresponding physical signal providing prompting effect or inhibiting effect to the main task;
a second acquiring unit, configured to acquire feedback response information of a subject corresponding to the main task and the auxiliary task.

8. The device for testing user experience as described in claim 7, wherein the task releasing unit is specifically configured to:
release, based on a predetermined releasing rule or randomly, a main task and an auxiliary task according to the test indicating information, wherein a number of the main task is larger than a number of the auxiliary task.

9. The device for testing user experience as described in claim 7, wherein
the physical signal is a vibration signal.

10. The device for testing user experience as described in claim 7, wherein
the feedback response information of the subject comprises response time.

11. The device for testing user experience as described in claim 10, wherein the feedback response information of the subject further comprises at least one of pressing pressure and pressing time.

12. The device for testing user experience as described in claim 10, wherein the auxiliary task comprises a first auxiliary task and a second auxiliary task, a physical signal of the first auxiliary task and a physical signal of the second auxiliary task have at least one difference parameter; and
the device for testing user experience further comprises:
a perception situation determining unit, configured to, after the second acquiring unit acquires the feedback response information of the subject, determine perception situation of the subject on the difference parameter according to the feedback response information of the subject.
